(19) United States
(12) Patent Application Publication
(10) Pub. No.: US 2025/0154608 A1
(43) Pub. Date: May 15, 2025

(54) SYSTEM AND METHODS FOR EARLY DETECTION OF VIABLE MICROORGANISMS

(71) Applicant: Frontline Biotechnologies, d.b.a. PureBioX, Saint Paul, MN (US)

(72) Inventors: Abdennour Abbas, Falcon Heights, MN (US); Akli Zarouri, Saint Paul, MN (US)

(73) Assignee: Frontline Biotechnologies, d.b.a. PureBioX, Saint Paul, MN (US)

(21) Appl. No.: 18/943,470

(22) Filed: Nov. 11, 2024

Related U.S. Application Data
(60) Provisional application No. 63/597,400, filed on Nov. 9, 2023.

(51) Int. Cl. C12Q 1/689; C12Q 1/6806; C12Q 1/6809; C12Q 1/6874

(52) U.S. Cl. CPC C12Q 1/689; C12Q 1/6806; C12Q 1/6809; C12Q 1/6874

(57) ABSTRACT

The present disclosure, in one embodiment, relates to devices, systems and methods that can be used to detect viable microorganisms during the lag phase (the first 1-60 min) of cell growth, thus overcoming the need to wait for days or weeks for cell enrichment and growth to achieve visible colonies. This diagnostic method relies on the qualitative and quantitative detection and profiling (fingerprint detection) of transcriptional and metabolic changes that follow the inoculation of microorganisms (bacteria, fungi, viruses, etc.) into a specific fresh medium, which represent the earliest stages of bacterial growth during the lag phase. This new method is named “Lag Phase-based Microbial Identification”

### CROSS REFERENCE TO RELATED APPLICATION

This application claims priority to U.S. Provisional Application No. 63/597,400, entitled “Devices and Methods for Early Detection of Viable Microorganisms,” which was filed on Nov. 9, 2023, and which is hereby incorporated herein in its entirety.

### FIELD OF THE INVENTION

The present disclosure relates to systems and methods for detecting viable microorganisms during the lag phase (1-60 minutes) of cell growth that does not require the need to wait for days or weeks for cell enrichment to achieve visible colonies.

### BACKGROUND OF THE INVENTION

Despite the availability of molecular and genomic diagnostic tools, the detection, and identification of fungi in different industries still relies heavily on cell culture enrichment and examination. Compared to techniques such immunoassays, PCR or sequencing, fungal culture has several attractive advantages, for example, cell growth can confirm cell viability, which can be critical information in some applications. Further, in some cases fungal culture does not require specialized equipment, it can assess antifungal susceptibility, and fungal culture can be more accommodating, less complex, and less expensive when detecting co-infections with different pathogens.

However, fungal culture can be difficult to grow, and/or it can be difficult to identify by observation certain microbial species, which tends to result in high false negatives for some infections. Furthermore, microbial cultivation can be time consuming, requiring days to weeks to achieve a visible colony, which invariably delays diagnosis and decision making, leading to delays in treatments and an increase in hospitalization costs. For example, as may be seen in FIG. 1, fungal culture 100 may include culturing 104 a sample 102 and waiting days to weeks for the exponential growth phase 106. Once complete, the cells may be observed under microscope 108, and/or subjected to microbial DNA amplification 110, and/or DNA amplicon visualization or sequencing 112.

Thus, there is an urgent need for novel microbial identification tools that can enable rapid and broad-spectrum diagnostics with high specificity and sensitivity, and that can be operated and interpreted by inexperienced users with minimal training.

### BRIEF SUMMARY OF THE INVENTION

The present disclosure, in one embodiment, relates to a method to detect viable microorganisms in a sample, the method includes identifying the transcriptome of the lag phase of microbial growth of the sample during the first sixty minutes of exposure of the sample to a growth medium, wherein the transcriptome includes coding RNA and non-coding RNA. In some embodiments, the coding RNA is messenger RNA. In some embodiments, the non-coding RNA is transfer RNA or ribosomal RNA. In some embodiments, the transcriptome identification occurs within the first thirty minutes of exposure of the sample to the growth medium.

In some embodiments, the grown cells are lysed to extract RNA transcripts; and specific primers target the RNA transcripts of the genes encoding transcription, translation, iron-sulfur protein assembly, nucleotide metabolism, Lipopolysaccharide biosynthesis, and aerobic respiration, to initiate nucleic acid amplification and subsequent detection of the amplicons specific to at least one existing microbial community in the culture medium.

In some embodiments, the transcriptome identification occurs within the first twenty minutes of exposure of the sample to the growth medium. In some embodiments, the grown cells are lysed to extract RNA transcripts; and the method also includes using at least one specific primer targeting the RNA transcripts to initiate nucleic acid amplification and subsequent detection of an amplicon.

In some embodiments, the at least one specific primer targets the RNA transcripts of an inorganic phosphorous uptake gene. In some embodiments, the inorganic phosphorous uptake gene is pstSCAB or phoBR.

In some embodiments, the at least one specific primer targets the RNA transcripts of an iron uptake gene. In some embodiments, the iron uptake gene is entB, fep, or iro operons.

In some embodiments, the at least one specific primer targets the RNA transcripts of an RNA polymerase encoding gene. In some embodiments, the RNA polymerase encoding gene is rpoABCZ or hepA.

In some embodiments, the method also includes after extracting the RNA transcripts, using RNA sequencing to identify RNA transcript fingerprints that are used to identify specific microorganisms after comparison to a database.

The present disclosure in one embodiment relates to a method to detect viable microorganisms in a sample, the method comprising: exposing the sample of a microorganism to a specific cell culture medium for up to 60 minutes; lysing the exposed cells to extract RNA transcripts that are then subjected to nucleic acid amplification; and performing RNA sequencing. In some embodiments, the RNA sequencing detection is by RNA amplification. In some embodiments, the RNA sequencing detection is by transcript fingerprint identification.

The present disclosure in one embodiment relates to a method to detect viable microorganisms in a sample, the method comprising: exposing the sample of a microorganism to a specific cell culture medium for up to 60 minutes, wherein the specific culture medium contains dyes or chemical markers that change color due to a change in an iron, phosphate, calcium, manganese, or sulfur content in the culture medium; detecting changes in dye, chemical, or biological marker properties; and performing microbial identification. In some embodiments, detecting changes in the dye, chemical, or biological marker properties is performed by spectrometer or spectrophotometer.

In some embodiments, the microbial identification is performed by analyzing metabolic fingerprints of iron, phosphate, or sulfur uptake and comparison to a database.

While multiple embodiments are disclosed, still other embodiments of the present disclosure will become apparent to those skilled in the art from the following detailed description, which shows and describes illustrative embodiments of the disclosure. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the disclosure will be better understood from the following description taken in conjunction with the accompanying Figures, in which:

FIG. 1 is a block diagram of a prior art method of cell culture.

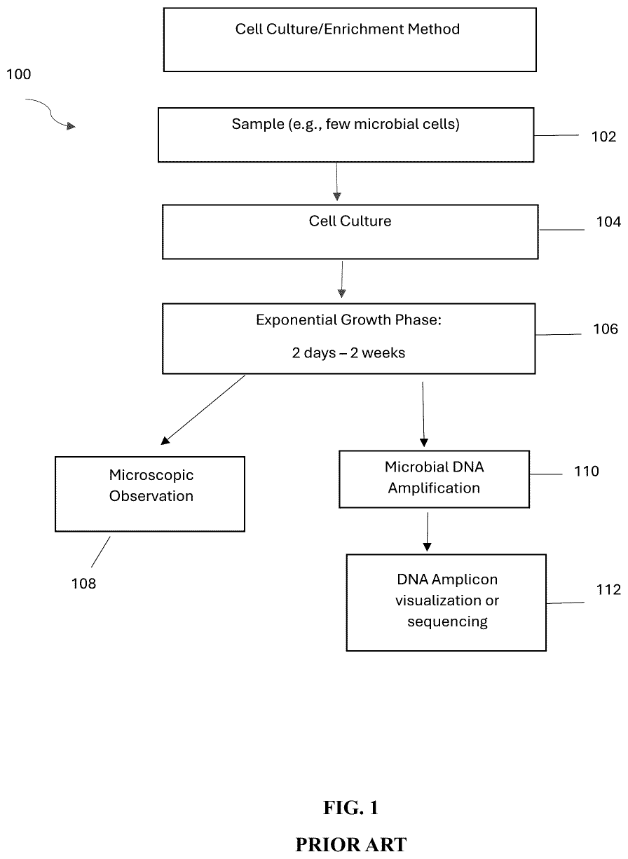

FIG. 2 is a block diagram of a method for lag phase-based transcriptional microbial identification, according to some embodiments of the present disclosure.

FIG. 3 is a block diagram of a method for lag phase-based metabolic microbial identification, according to some embodiments of the present disclosure.

### DETAILED DESCRIPTION

The present disclosure relates to systems and methods that can be used to detect viable microorganisms during the “lag phase” (the first 1-60 min, for example) of cell growth, thus overcoming the need to wait for days or weeks for cell enrichment and growth to achieve visible colonies. Diagnostic systems and methods of the present disclosure rely in some embodiments on qualitative and quantitative detection and profiling (fingerprint detection, for example) of transcriptional and metabolic changes that follow the inoculation of microorganisms (such as for example, but not limited to bacteria, fungi, viruses) into a specific fresh medium, which represent the earliest stages of bacterial growth during the lag phase. Embodiments of the present disclosure in some cases may be referred to as “Lag Phase-based Microbial Identification.” Systems and methods of the present disclosure, including Lag Phase-based Microbial Identification may have a profound impact in a number of different fields for a number of different, and in some cases related reasons. More particularly, invasive fungi identification represents an enabling market that has an impact on several sectors, including, but not limited to healthcare, military, food, and agriculture.

Public Health: Fungal infections caused over 7,000 deaths in the US during 2021 and represent a significant economic burden resulting in over $11.5 billion during 2019, and $7.5 billion in direct medical costs alone. (Benedict et li., 2022, doi: 10.1093/ofid/ofac097). Fungi that may cause serious infections in humans include, but are not limited to, yeasts, such as Candida, Cryptococcus, and Pneumocystis spp.; and molds, such as Aspergillus spp. Early identification and surveillance of fungal infection can be critical given the potential for severe infection and/or death resulting from infection. This may be particularly critical for people who are immunocompromised as a result of having or having had an organ or stem cell transplantation, cancer, from taking immunosuppressive medications; and/or from having a critical illness, for example. For example, patients with COVID-19-associated fungal infections have been reported to have higher (48.5%) in-hospital mortality rate. (Benedict et li., 2022, doi: 10.1093/ofid/ofac097).

Military Applications: U.S. combat injuries received by military personnel during recent wars have demonstrated that Invasive Fungal Wound Infections (IFI) represent a significant health and economic burden and shall continue to remain a threat for future modern warfare. Combat-related IFIs are associated with high mortality rates following severe blast trauma, that can be as high as 38%, depending on underlying conditions and how the disease was obtained. (Rodriguez et al., 2022, doi: 10.1093/milmed/usab074). Because of this, combat-related IFIs have been identified as a priority issue within the Department of Defense (DoD) Joint Trauma System (JTS), the Infectious Disease Clinical Research Program (IDCRP), and the Trauma Infectious Disease Outcomes Study (TIDOS). (Rodriguez et al., 2022, doi: 10.1093/milmed/usab074).

Plant Health and Agriculture: PureBioX interviews with managers of plant disease diagnostic clinics in the United States during the I-Corps program revealed that up to 70% of samples received for testing were infected by fungi. (NSF I-Corps program report, 2023). Fungi and oomycetes can destroy a third of all food crops each year globally that had it not been destroyed, would be sufficient to feed 600 million people. (Davies et al., 2021, doi: 10.1016/j.fbr.2021.01.003). Some fungi, such as the rice pathogen Magnaporthe oryzae, can result in yield losses of up to 100 (Davies et al., 2021, doi: 10.1016/j.fbr.2021.01.003).

Forest Health: Invasive fungi are one of the deadliest microorganisms for trees and forests worldwide. The impact of invasive fungi is expected to increase due to climate change. (Siedel et al., 2024, https://doi.org/10.1016/S2666-5247(24)00039-9).

Rapid approaches for early detection of forest pathogens are sorely needed. In the United States, approximately $2.1 billion in forest products may be lost each year as a result of alien forest pathogens, mostly fungi. (Pimentel et all., 2001, Agriculture, Ecosystems and Environment 84 (2001) 1-20).

For example, the invasive fungus Ceratocystis fagacearum, which is the causal organism of oak wilt that threatens the health of oak trees in the United States, damages 77 million acres of total forested land and 14% of the national timberland. (USDA Report, https://www.nrs.fs.usda.gov/pubs/gtr/gtr_nc228.pdf). The disease is currently known to exist in 24 states in the United States. (USDA Report, https://www.nrs.fs.usda.gov/pubs/gtr/gtr_nc228.pdf). A Minnesota study on oak trees published in 2011 revealed that around 266,000 trees were infected by the oak wilt fungus, resulting in a discounted tree removal cost of $18-60 million ($400-500 per tree). (Haight, 2011, doi: 10.1007/s00267-011-9624-5). The fungus can be in a tree for two to three weeks without leaf symptoms appearing. Preventing spread of the disease requires rapid, pre-symptomatic detection of the causal fungal agent, which would help stop the spread of the disease and minimize the inherent cost for municipalities, counties, the state and/or private property owners.

Food industry: Fungi represent a major source of worldwide food poisoning and spoilage and can have major socioeconomic and health impacts, affecting food safety and security. The Food and Agriculture Organization (FAO) of the United Nations estimates that 25% of the world's food crops are affected by mycotoxins produced by certain fungi. (USDA Report, https://www.fsis.usda.gov/food-safety/safe-food-handling-and-preparation/food-safety-basics/molds-food-are-they-dangerous#:˜:text=There %20are %20many %20of %20them,the %20most %20notorious %2 0are%20aflatoxins.) If fungal detection could be performed in less than two allowing relevant actors to make informed decisions before releasing a product would be hugely significant in reducing food spoilage and ensuring food safety. According to the Centers for Disease Control and Prevention (CDC), each year roughly 48 million people (1 in 6 Americans) get sick, 128,000 are hospitalized, and 3,000 die of foodborne diseases. (CDC Report, https://www.cdc.gov/foodborneburden/2011-foodborne-estimates.html). A 2010 report of the Pew Charitable Trusts estimates that foodborne diseases cost the U.S. roughly $152 billion per year in medical bills and lost workdays. (Pew Charitable Foundation report, https://www.pewtrusts.org/en/research-and analysis/reports/0001/01/01/healthrelated-costs-from-foodborne-illness-in-the-united-states). Early and rapid fungi screening can prevent food poisoning, while reducing food loss by allowing early intervention to delay deterioration. The global market for Food Safety Testing Services is projected to reach $19 billion by 2022. (Fortune Business Insight Report (https://www.fortunebusinessinsights.com/food-safety-testing-market-108286#). This is driven by concerns over foodborne illnesses, and food spoilage, and the increasingly stringent government regulations on food safety.

A common feature in all these industries is the fact that the detection of invasive fungi in food, agricultural samples or human specimens still relies on cell culture that requires 2 days to 2 weeks and sometime more to achieve a visible identifiable colony. The delay in diagnostics leads to the spread of disease and an increase in human illness and related economic costs. In contrast to existing culture methods that take up to weeks, systems and methods of the present disclosure in some embodiments may take as little minutes, for example, from 6-20 minutes, or two hours total, in some embodiments. Whereas the sensitivity for prior art culture may be around 40-90% with an LOD of 10²-10³ cfu/g, embodiments of the systems and methods of the present disclosure may have a sensitivity of greater than 90% with an LOD of less than 100 cfu/g. And whereas prior art culture may be based on microscopic detection of fungi and bacteria, embodiments of systems and methods of the present disclosure may be based on molecular detection of fungi and bacteria.

Systems and methods of embodiments of the present disclosure may detect the presence of viable microorganisms during the lag phase by taking advantage of changes that happen during the first 4-20 minutes of the early stage of growth immediately after inoculation of the microorganism into a new culture medium. These changes include significant genetic changes, particularly including transcription and RNA production and significant metabolic and energetic changes, including ionic changes and metal uptake. According to systems and methods of the present disclosure, a variety of RNA targets can be detected by genetic amplification techniques such as RT-PCR, RT-LAMPS or sequencing, and metabolic targets can be identified with spectroscopic or spectrophotometric methods and specific chemical or biological markers or reporters.

In some embodiments of the present disclosure microbial identification may be determined through the detection of transcriptomic changes during the lag phase as is discussed and shown in Table 1 below. During the lag phase (post-inoculation), microbial cells can produce 5-50 ug of RNA. This is the result of large-scale transcriptional changes occurring during the lag phase leading to a change in expression levels of over 1,100 genes within four minutes of inoculation, 1,741 genes within 20 minutes, and around 2,657 genes (more than half of the genome) within 40 minutes.

These changes include 1-to-70 fold upregulation of genes involved in transcription, nucleotide metabolism, lipopolysaccharide and fatty acid biosynthesis, genes encoding aminoacyl-tRNA synthetases and ribosomal proteins, and RNA polymerase, genes encoding the terminal cytochrome oxidase bo (cyoABCD) were upregulated between 4-and 10-fold.

One of the most highly regulated genes is pstSCAB, upregulated up to 70-fold within 4 minutes into the lag phase. This gene encodes the major ABC transporter for inorganic phosphate Pᵢ uptake, and is PhoBR-regulated. The rapid induction of phoBR and pstSCAB is consistent with a requirement for the uptake of inorganic phosphate during lag phase. Studies on other species such as Bacillus licheniformis also identified upregulation of genes involved in phosphate transport during lag phase, suggesting that phosphate uptake may be a universal requirement during lag phase.

TABLE 1
Regulated genes during the lag phase as targets for microbial detection (compiled
from Rolfe et al., 2012, DOI: https://doi.org/10.1128/JB.06112-11)

| | | | |
|---|---|---|---|
| Gene/RNA | | | |
| transcript | Role | Expression/Detection | Upregulation |

Upregulated genes

| | | | | |
|---|---|---|---|---|
| pstSCAB | Pi uptake | 4 | min | 70-fold |
| phoBR | Pi uptake | 4 | min | |
| cyoABCD | terminal cytochrome oxidase | 4 and 20 | min | |
| pgi | phosphog- lucose isomerase | 4 and 20 | min | |
| tpiA | triose phosphate isomerase | 4 and 20 | min | |
| eno | enolase | 4 and 20 | min | |
| ndh | encoding NADH dehydrogenases | 4 and 20 | min | 5-fold |
| fba | fructose-bisphosphate aldolase | 4 and 20 | min | |
| nine genes: entB, | Iron uptake | 4 and 20 | min | |
| fep, iro operons | | | | |
| Four genes: isc, suf | Fe-S cluster synthesis | 4 and 20 | min | |
| operons | | | | |
| | RNA of aminoacyl-tRNA | 20 | min | |
| | synthetases | | | |
| | Fatty acid biosynthesis | | | |
| ahpC | OxyR regulon | 4 and 20 | min | |
| acrB | SoxS regulon | 4 | min | |
| rpoABCZ, hepA | RNA polymerase | 20 | min | |
| rpl, rpm, rps | ribosomal proteins | 20 | min | |
| operons | | | | |

Down-regulated genes

| | | | |
|---|---|---|---|
| FbaB | fructose-bisphosphate aldolase | 20 | min |
| pfkB | phosphofructokinase | 20 | in |

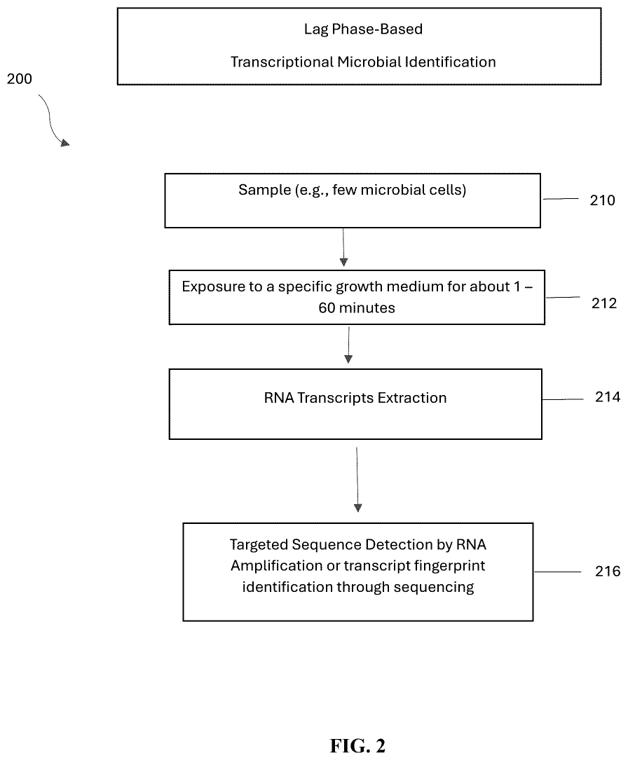

In some embodiments of the present disclosure, as may be seen in FIG. 2, systems and methods to detect viable microorganisms include lag phase-based microbial identification 200. Embodiments of systems and methods of lag phase-based microbial identification 200 may include taking a sample 210 and exposing the microorganisms to a specific cell culture medium for 1-60 minutes 212, for example, and in some embodiments for 4-30 minutes, for example. The cells may then be lysed to extract the RNA transcripts 214. Examples of cell culture medium that may be used include, but are not limited to, Luria-Bertani (LB) broth and agar, Sabouraud Dextrons Agar (SDA), Potato Dextrose Agar (PDA), Malt Extract Agar (MEA) and Czapek Dox Agar. In certain embodiments, these media may be modified by adding dyes and growth factors, such as for example, but not limited to biotin, thuamine, and riboflavin, or aminoacids and nucleotides. Next according to some embodiments, microbial identification may be determined, including in one of the following ways:
(1) by using specific primers targeting the RNA transcripts of the genes encoding transcription, translation, iron-sulfur protein assembly, nucleotide metabolism, Lipopolysaccharide biosynthesis, and aerobic respiration, to initiate nucleic acid amplification and subsequent detection of the amplicons specific to the existing microbial communities in the culture medium. Examples of primers that may be used include, but are not limited to, complementary sequences of RNA transcripts of the genes pstSCAB, phone, EntB, fep, iro operons and other genes coding for phosphate and iron uptakes;
(2) by extracting the RNA transcripts, then using genome sequencing of the RNA transcripts to identify transcriptomic fingerprints of existing microbial communities in the culture medium 216; and
(3) by extracting the RNA transcripts, then using nucleic acid amplification using RT-PCR or RT-LAMP, for example, to amplify RNA transcripts expressed during the lag phase, including but not limited to those encoding for the inorganic phosphorus uptake genes, such as pstSCAB and phoBR, iron uptake genes, such as but not limited to, entB, fep, and iro operons, the RNA polymerase encoding genes, including, but not limited to rpoABCZ, hepA and other RNA transcripts involved in transcription, translation, iron-sulfur protein assembly, nucleotide metabolism, lipopolysaccharide biosynthesis, and aerobic respiration. This may then be followed by RNA sequencing and identification RNA transcript fingerprints that will be compared to a database to identify specific microorganisms.

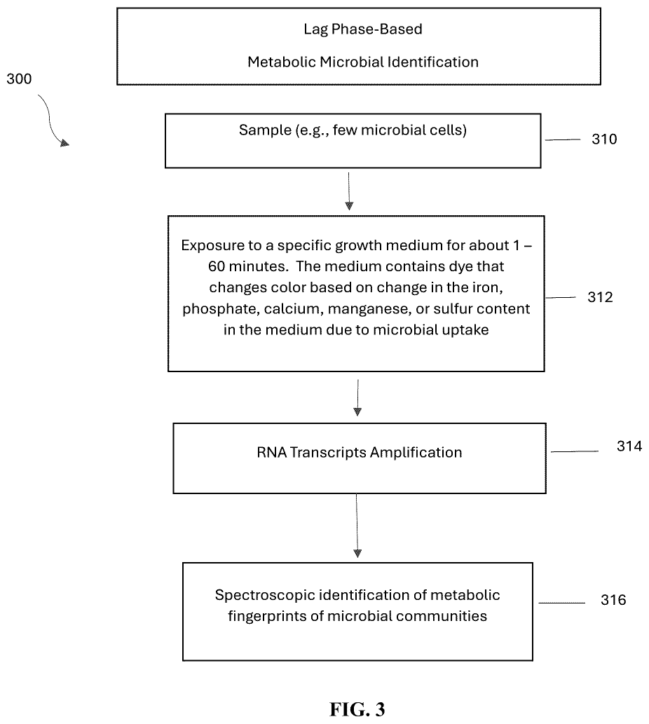

In other embodiments of systems and methods of the present disclosure microbial identification may be determined through the detection of metabolic changes during the lag phase 300, as shown in FIG. 3. During the early stages of the lag phase (for example, from 1-60 minutes), there is a significant increase in uptake of some elements and nutrients by the microbial cells due to high transcriptomic activity. These changes include:

(1) Phosphate uptake during the lag phase: Phosphate is an essential mineral for bacterial growth, required as an integral component of membrane phospholipids, nucleic acids, and nucleotides, and for phosphorylation events within cells. Uptake of phosphate increases during lag phase to support these processes leading to early upregulation of genes involved in phosphate uptake pathways. Three forms of phosphate can be utilized: inorganic phosphate (Pi), organophosphates, and phos-phonates. Phosphonates and Pi are taken up directly, while most organophosphates are degraded in the periplasm to yield Pi, which is then transported into the cell.

(2) Iron uptake during lag phase: Iron accumulation increases during the lag phase, with the highest cellular concentration (4.1×10-18 moles per cell) during the earliest stages of lag. Iron accumulation correlates with the expression of the dedicated iron transport machinery, which increases during the lag phase. Iron uptake increases during the earliest stages of growth because it is needed for the assembly of iron cofactors and Fe—S clusters that are associated with essential metabolic pathways.

(3) Uptake of other elements: Uptake of other elements such as calcium and manganese also increase during the early stage of the lag phase.

In some embodiments of the present disclosure, as may be seen in FIG. 3, microbial detection and identification during the lag phase includes taking a sample 310 and exposing the microorganisms to a specific cell culture medium for, in some embodiments about 1-60 minutes, and in some embodiments, from about 4-30 minutes 312. The culture medium may contain colored or colorless dyes or other chemical or biological markers that change color due to a change in the iron, phosphate, calcium, manganese, or sulfur content in the culture medium due to uptake by existing microorganisms 312. Examples of these growth media include, but are not limited to: LuriaBertani (LB) broth, Tryptic soy broth (TSB), Trypric soy agar (TSA), M9 minimal medium, Mannitol salt agar, MacKomkey agar, agar media, and yeast extract peptone dextrose (YEPD). RNA transcript amplification 314 may be performed. Changes in the dye or chemical or biological marker properties, such as color may be detected and measured with a spectrometer or spectrophotometer 316. Microbial identification may then be performed by the analysis of metabolic fingerprints of iron, phosphate, or sulfur uptake, for example, and then using a database for comparison to make the identification 316.

Embodiments of systems and methods of the present disclosure have been described in detail by way of illustration and example for purposes of clarity and understanding. However, it will be apparent that certain changes and modifications may be made within the spirit and scope of the present disclosure and are thereby within the scope of the present disclosure.

What is claimed is:

1. A method to detect viable microorganisms in a sample, the method comprising:
identifying the transcriptome of the lag phase of microbial growth of the sample during the first sixty minutes of exposure of the sample to a growth medium,
wherein the transcriptome includes coding RNA and non-coding RNA.

2. The method of claim 1, wherein the coding RNA is messenger RNA.

3. The method of claim 1, wherein the non-coding RNA is transfer RNA or ribosomal RNA.

4. The method of claim 1, wherein the transcriptome identification occurs within the first thirty minutes of exposure of the sample to the growth medium.

5. The method of claim 4, wherein the grown cells are lysed to extract RNA transcripts; and
further comprising, using specific primers targeting the RNA transcripts of the genes encoding transcription, translation, iron-sulfur protein assembly, nucleotide metabolism, Lipopolysaccharide biosynthesis, and aerobic respiration, to initiate nucleic acid amplification and subsequent detection of the amplicons specific to at least one existing microbial community in the culture medium.

6. The method of claim 5, wherein the transcriptome identification occurs within the first twenty minutes of exposure of the sample to the growth medium.

7. The method of claim 1, wherein the grown cells are lysed to extract RNA transcripts; and
further comprising using at least one specific primer targeting the RNA transcripts to initiate nucleic acid amplification and subsequent detection of an amplicon.

8. The method of claim 7, wherein the at least one specific primer targets the RNA transcripts of an inorganic phosphorous uptake gene.

9. The method of claim 8, wherein the inorganic phosphorous uptake gene is pstSCAB or phoBR.

10. The method of claim 7, wherein the at least one specific primer targets the RNA transcripts of an iron uptake gene.

11. The method of claim 10, wherein the iron uptake gene is entB, fep, or iro operons.

12. The method of claim 7, wherein the at least one specific primer targets the RNA transcripts of an RNA polymerase encoding gene.

13. The method of claim 12, wherein the RNA polymerase encoding gene is rpoABCZ or hepA.

14. The method of claim 7, further comprising after extracting the RNA transcripts, using RNA sequencing to identify RNA transcript fingerprints that are used to identify specific microorganisms after comparison to a database.

15. A method to detect viable microorganisms in a sample, the method comprising:
exposing the sample of a microorganism to a specific cell culture medium for up to 60 minutes;
lysing the exposed cells to extract RNA transcripts that are then subjected to nucleic acid amplification; and
performing RNA sequencing.

16. The method of claim 15, wherein the RNA sequencing detection is by RNA amplification.

17. The method of claim 15, wherein the RNA sequencing detection is by transcript fingerprint identification.

18. A method to detect viable microorganisms in a sample, the method comprising:
exposing the sample of a microorganism to a specific cell culture medium for up to 60 minutes,
wherein the specific culture medium contains dyes or chemical markers that change color due to a change in an iron, phosphate, calcium, manganese, or sulfur content in the culture medium;
detecting changes in dye, chemical, or biological marker properties; and
performing microbial identification.

19. The method of claim 18, wherein detecting changes in the dye, chemical, or biological marker properties is performed by spectrometer or spectrophotometer.

20. The method of claim 18, wherein microbial identification is performed by analyzing metabolic fingerprints of iron, phosphate, or sulfur uptake and comparison to a database.
